(19) United States
(12) Patent Application Publication
(10) Pub. No.: US 2012/0213754 A1
(43) Pub. Date: Aug. 23, 2012

(54) Method of Preconditioning of Cell Suspensions

(75) Inventor: John R. Chapman, Sacramento, CA (US)

(73) Assignee: STEM CELL PARTNERS LLC, Sacramento, CA (US)

(21) Appl. No.: 13/398,739

(22) Filed: Feb. 16, 2012

Related U.S. Application Data
(60) Provisional application No. 61/446,007, filed on Feb. 23, 2011.

(51) Int. Cl. A61K 35/14; A61K 35/18; A61K 35/12; A61K 35/16; C12N 5/071; C12N 5/0775

(52) U.S. Cl. 424/93.71; 435/2; 435/375; 424/93.7; 424/93.72; 424/93.73

(57) ABSTRACT

An apparatus for the preparation of cells for implantation into a living body is disclosed. The apparatus comprises a vessel substantially impermeable to gaseous oxygen; and fluid within said vessel, the fluid having a maximal dissolved oxygen capacity substantially equivalent to normal saline yet having a hypoxic oxygen concentration between about 0% to about 5% of said maximal dissolved oxygen capacity. The bag oxygen concentration level remains low when the apparatus is stored at 22°-25° C. in normal atmospheric conditions for a period of at least 30 days. The present invention simplifies the process of achieving donor cell hypoxic preconditioning for cell implantation, and may be used to bathe said cells to be transplanted for a sufficient time to activate the hypoxic metabolic pathway.

### RELATED APPLICATION

This application claims priority from the U.S. provisional application with Ser. No. 61/446,007, which was filed on Feb. 23, 2011. The disclosure of that provisional application is incorporated herein as if set out in full.

### BACKGROUND

1. Field of the Invention

The present invention relates generally to the preparation of living cells for transplantation, and specifically to an apparatus and method for contacting living cells with fluids manufactured to have a low dissolved oxygen concentration, thereby activating the hypoxic cellular pathway within said living cells.

2. Background of the Invention

Mammalian cells are required for many scientific and medical procedures, such as cell transplantation, tissue culture expansion and other diagnostic tests. There are many methods for preparing a concentrate of such cells. One method is disclosed in U.S. Pat. No. 7,291,450 to Sowemimo-Coker et al, which describes methods and compositions regarding the preparation of a cell concentrate by filtration.

Stem cell concentrates are generally obtained through the centrifugation of bone marrow aspirate, cord blood, peripheral blood, or lipoaspirate or a mixture thereof. Other procedures are possible. By preconditioning the cells from which stem cell concentrate is to be obtained, the success rate of the procedures can be increased. One conventional preconditioning method exploits certain effects of hypoxia on cells. Hypoxia may be broadly defined as the state in which oxygen (O₂) metabolic demand exceeds supply. Mammals require molecular O₂ for essential metabolic processes including oxidative phosphorylation in which O₂ serves as an electron acceptor during ATP formation. Systemic, local, and intracellular homeostatic responses elicited by hypoxia include among others erythropoiesis by individuals who are anemic or at high altitude, neovascularization in ischemic myocardium, and glycolysis in cells cultured at reduced O₂ tension. These adaptive responses either increase O₂ delivery or activate alternate metabolic pathways that do not require O₂. Hypoxia-inducible gene products that participate in these responses include erythropoietin (EPO), vascular endothelial growth factor (VEGF), and glycolytic enzymes.

The molecular mechanisms that mediate genetic responses to hypoxia have been extensively investigated for the EPO gene, which encodes a growth factor that regulates erythropoiesis and thus blood O₂ carrying capacity. Cis-acting DNA sequences required for transcriptional activation in response to hypoxia were identified in the EPO S'-flanking region and a trans-acting factor that binds to the enhancer, a hypoxia-inducible factor 1 (HIF-1), fulfilled criteria for a physiological regulator of EPO transcription. In particular, inducers of EPO expression (1% O₂, cobalt chloride [CoCl₂], and desferrioxamine [DFX]) also induced HIF-1 DNA binding activity with similar kinetics. EPO expression is cell type specific, but induction of HIF-1 activity by 1% O₂, CoCl₂, or DFX was detected in many mammalian cell lines.

HIF-1 protein is a heterodimeric mammalian transcription factor expressed uniquely in response to physiologically relevant levels of hypoxia. HIF-1 is a basic helix loop-helix protein that binds to cis-acting hypoxia-responsive elements of genes induced by hypoxia. HIF-1 DNA binding activity and HIF-1 protein concentration increase exponentially as cells are subjected to decreasing O₂ concentrations.

The hypoxia-inducible factor 1 alpha (HIF-1α) is the regulated member of the transcription factor heterodimer HIF-1 α/β. HIF-1α a binds to hypoxia-response elements (HREs) in the promoters of many genes involved in the adaptation to an environment of insufficient oxygen or hypoxia. Thus, HIF-1 constitutes the major molecular response to hypoxia. HIF-1 consists of two basic helix-loop-helix proteins (an oxygen-sensitive HIF-1 α subunit and an oxygen-insensitive HIF-1beta subunit) and three regulatory HIF subunits (HIF-1a, HIF-2 α, and HIF-3 α). HIF-1a is expressed ubiquitously, whereas the expression of HIF-2 α and HIF-3 α appears to be restricted to certain tissues. Regulation of HIF-1 activity is mediated by post-translational modification of the oxygen-dependent degradation domain (ODD) in the HIF-1 α subunit.

At oxygen levels above 5% of the maximum saturation of dissolved oxygen concentration of the fluid, hydroxylation of proline residues 402 and 564 in the ODD of HIF-1 α enables binding of the ubiquitination ligase von Hippel-Lindau tumor suppressor protein, which in turn leads to degradation of HIF-1 α by the proteasome. In contrast, at oxygen levels at or below 5% dissolved oxygen concentration, hydroxylation is inhibited leading to stabilization of HIF-1 α. Mammalian cells are thus able to sense oxygen tension and turn on a series of genes in response to the lack of oxygen. Expression of HIF-1 proteins and the level of HIF-1 DNA binding activity is known to undergo an exponential increase with decreasing percent dissolved oxygen concentrations for instance from ˜5% to 0% of the maximum dissolved oxygen concentration of the fluid, with half maximal response at ˜1.5% of the maximum dissolved oxygen concentration of the fluid, which is the critical range of oxygen partial pressures measured for tissues in vivo. Hypoxia therefore has two independent effects on HIF-1 α activity: (1) hypoxia increases the steady-state levels of HIF-1 α protein by stabilizing it (i.e. decreasing its degradation); and (2) hypoxia increases the specific transcriptional activity of the protein (i.e. independent of the protein concentration).

HIF-1 is also involved in immune reactions. Leukocyte trafficking, an event which plays a central role in fundamental functions of multicellular organisms, including tissue remodeling, defense, and pathology, is orchestrated by a superfamily of small proteins termed chemokines, which are essential players in immune and inflammatory reactions as well as in infection. Immunomodulatory peptides, including interleukin-1 (IL-1) and tumor necrosis factor-alpha (TNF-alpha), stimulate HIF-1 dependent gene expression even in normoxic cells. Both the hypoxic and the cytokine-induced activation of HIF-1 involve the phosphatidylinositol-3-kinase (PI3K) and the mitogen-activated protein kinase (MAPK) signaling pathways. In addition, heat shock proteins (HSP) and other cofactors interact with HIF-1 subunits. HIF-1 increases the transcription of several genes for proteins that promote blood flow and inflammation, including vascular endothelial growth factor (VEGF), heme oxygenase-1, endothelial and inducible nitric oxide synthase (NOS) and cyclooxygenase-2 (COX-2). Exposure to hypoxia significantly enhances CXCR4 expression in several cell types including blood leukocytes to hypoxia induced CXCR4 expression.

Migration of hematopoietic stem cells through the blood, across the endothelial vasculature to different organs and to their bone marrow (BM) niches, requires active navigation, a process termed homing. Homing is the first and most essential step in clinical stem cell transplantation. Homing is a fairly rapid process (generally measured in hours and no longer than 1-2 days) in which circulating hematopoietic cells actively cross the blood/BM endothelium barrier and lodge at least transiently in the BM compartment by activation of adhesion interactions prior to their proliferation. Homing is thought to be a coordinated, multistep process, which involves signaling by stromal-derived factor 1 (SDF-1) and CXCR4 cell surface expression as well other adhesion molecules.

Mesenchymal stem cells (MSC) are adult multipotent cells found in bone marrow, cord blood, adipose tissue, and other adult tissues. MSCs are known to improve regeneration of injured tissues in vivo, but the mechanisms for this process remain unclear. It is known that hypoxic preconditioning increases expression of pro-survival and proangiogenic factors including HIF-1, angiopoietin-1, vascular endothelial growth factor and its receptor, Flk-1, erythropoietin, Bcl-2, and Bcl-xL. Further, cell death of hypoxic stem cells and caspase-3 activation in these cells is significantly lower compared with that in normoxic stem cells both in vitro and in vivo. Transplantation of hypoxic versus normoxic mesenchymal stem cells after myocardial infarction results in an increase in angiogenesis, as well as enhanced morphologic and functional benefits of stem cell therapy. Typically, MSCs are cultured under ambient, or normoxic, conditions and in an exemplary case at 21% dissolved oxygen concentration. However, the physiological niches for MSC in the bone marrow and other sites have much lower oxygen tension. Thus, when used as a therapeutic tool to repair tissue injuries, MSCs cultured in standard conditions must adapt from 100% of the dissolved oxygen capacity of the culture fluid, which can be 6 to 8 mg/L in culture to less than 1 mg/L in the ischemic tissue. To enhance their tissue regenerative potential, the preculturing of human bone marrow-derived MSCs in hypoxic conditions has been studied. In these studies, conducted on mice, intra-arterial injection of MSCs cultured in either normoxic or hypoxic conditions 24 hours after surgical induction of hind limb ischemia enhanced revascularization compared with saline controls. However, restoration of blood flow was observed significantly earlier in mice that had been injected with hypoxic preconditioned MSCs. It can be assumed that preculturing MSCs under hypoxic conditions prior to transplantation thus improves their tissue regenerative potential.

The pharmacologic activation of the HIF-1 complex can be desirable in ischemic and inflammatory disorders. All these genes also respond like EPO, to cobalt ions and iron chelators including desferrioxamine, suggesting a common mechanism for oxygen sensing and gene activation. Measurement of HIF-1α may be achieved by commercial kits such as those manufactured by R&D Systems, Minneapolis, Minn. as covered by U.S. Pat. Nos. 5,882,914, 6,020,462, and 6,222,018, the entire contents of which are by reference included herein. An example of a hypoxic preconditioning apparatus is the Oxycycler C42 commercialized by BioSpherix Corp., Lacona, N.Y.

Oxygen in fluids such as water, blood, or tissue culture may be expressed in various ways, including partial pressure of oxygen (pO₂) measured in mm of Hg (1 mmHg=133 Pa), percent of maximum (saturated) dissolved oxygen of a fluid, or mg/L (ppm) of dissolved oxygen. The amount of oxygen that may be dissolved in water is inversely proportional to water temperature, salinity, and altitude. At 25° C. and 1 atmosphere (760 mm of Hg), the maximum saturated oxygen concentration of water is 20.9% of 760 mm of Hg, which is equal to 159 mm of Hg. Under the same conditions, the maximum dissolved oxygen concentration is 8.52 mg/L (ppm), which represents 100% of the maximum dissolved oxygen concentration of water at that state.

According to Newby (Placenta (2005) 26:353-357), there are pitfalls for achieving the intended oxygen concentration when using atmospheric gas to achieve intended oxygen concentrations in tissue culture media fluid. Newby notes that cell culture models have been used to study the effect of oxygen on cell behavior in vitro; however, there is often little or no validation of oxygen levels in these cell culture systems. In Newby's study on this topic, dissolved oxygen levels in culture medium were first maintained in standard culture conditions (18% atmospheric O₂). On transfer to a low oxygen environment (2% O₂ atmosphere), oxygen levels decreased to 6-8% after 4 h and reached 2% only after 24 h according to FIG. 8.

A second experiment was performed by Newby. When nitrogen gas was bubbled through culture medium, dissolved oxygen levels fell from 18-20% to approximately 1.5% (11 mm of Hg) after 15 min and to 0% after 30 min. When pre-gassed culture medium (0%) was placed directly into the incubator with the 2% oxygen environment, oxygen levels in the culture medium equilibrated to approximately 2% after 1 h and remained at 2-3% (15-23 mm Hg) following overnight incubation.

A third experiment was performed by Newby. When culture medium pre-gassed with nitrogen to eliminate oxygen (0%) had been dispensed in ambient air, dissolved oxygen levels increased to 8.5% (65 mm Hg). Further incubation of the cell culture medium in a hypoxic environment of 2% oxygen for approximately 16 hours was required for dissolved oxygen level in the culture medium to decrease to 2% (15 mm Hg).

Thus, Newby reports that cultured cells placed in a low oxygen environment would be exposed to varying levels of oxygen before the desired level of oxygen exposure is reached. Newby concluded that his study highlights the importance of validation of oxygen levels and potential problems associated with in vitro studies on the regulatory effects of oxygen. Newby offered no other solutions to this problem.

There are many means for removing or reducing oxygen from aqueous solutions, including but not limited to bubbling nitrogen gas through the aqueous solution, as described in I. Butler, Talanta 41 (2): 211-215, 1994, and forced convection of a liquid through a capillary with a cell culture adhered to its sides, as described in Baumgardner et al., Respiratory Physiology & Neurobiology, Vol. 136 Issues 2-3, 16 Jul. 2003, Pages 131-139.

An example of design, materials and construction of oxygen impermeable containers is disclosed by U.S. Pat. No. 6,271,351 to Gawryl et al.

A meter suitable for measuring dissolved oxygen concentrations in the cell contacting fluid is the YSI Model 95 Handheld Dissolved Oxygen and Temperature System (YSI, Yellow Springs, Ohio) or commercially available oxygen sensors such as those manufactured by Oakton Instruments, Vernon Hills, Ill. The measurement of cellular HIF-1α may be achieved using ELISA methodologies such as commercial kits sold by R&D Systems, Minneapolis, Minn. (Catalog Number DYC1935-2).

Regenerative medicine is the use of cells as therapeutic agents to achieve desired clinical outcomes. Cells are injected into the body or applied to sites of wounds or injury to facilitate recovery or to enhance cosmetic appearance. The process of implanting cells by injection or topical application normally occurs in the operating room or in a patient treatment room. It is generally desirable to have a means to improve the clinical outcome achieved when cells are use for regenerative medicine applications. As described above, it is known in the prior art that one way of enhancing a cell's therapeutic potential is to activate the hypoxic response pathway.

Despite its benefit being known in the art for more than a decade, there remains a need for achieving a practical manner to activate the hypoxic response pathway in a clinical setting.

The best solutions disclosed or suggested by the prior art for the hypoxic preconditioning of donor cells are procedures that remain confined to the research laboratory. No solution has achieved clinical acceptance.

The present invention enables donor cell preconditioning to be performed at the point of care. Known methods for cell preconditioning are unsuitable for use in the operating room. An example of a device unsuitable for point of care donor cell precondition is the Oxycycler C42 commercialized by BioSpherix Corp., Lacona, N.Y., which is large, bulky, requires multiple gas cylinders and is generally beyond the capability of a physician or nurse to operate.

It is a first objective of the present invention to provide a reproducible method that does not rely on the use of complex, large, non-portable, labor intensive, expensive medical equipment that must be maintained and calibrated before each use.

It is a second objective to provide a method wherein the entire process of harvesting, conditioning and delivering the cells within the same surgical procedure can be accomplished.

It is a third objective to reduce the cost of preconditioning cells by eliminating the need for moving the cells into a GMP laboratory for processing.

It is a fourth objective to reduce regulatory hurdles required for implementation of the present invention by completely processing the cells within one surgical procedure.

It is a fifth objective of the present invention to reduce human error in the donor cell conditioning process.

### SUMMARY OF THE INVENTION

An apparatus for the preparation of cells for implantation into a living body is disclosed. The apparatus comprises a vessel substantially impermeable to gaseous oxygen; and fluid within said vessel, the fluid having a maximal dissolved oxygen capacity substantially equivalent to normal saline (approximately between 8-10 mg/L) and having a hypoxic oxygen concentration between about 0% to about 5% of said maximal dissolved oxygen capacity wherein said oxygen concentration is substantially stable when the apparatus is stored at 22°-25° C in normal atmospheric conditions for a period of at least 30 days. Normal saline is an aqueous solution of 0.9% sodium chloride (weight/volume).

The invention disclosed herein enables a health care worker such as a physician or nurse to prepare donor cells for subsequent transplantation by activating the hypoxic response pathway of those cells at the point of care. The present invention simplifies the process of achieving donor cell hypoxic preconditioning and makes it practical for routine clinical use. All of the various equipment from the prior art that would normally be used by a research laboratory to achieve this goal may be replaced in an exemplary embodiment of the current invention by a system comprising a bag of saline modified to have a low oxygen concentration as defined herein and packaged in a manner to maintain that low oxygen concentration for the life of the product, which may be at least 3 months and more preferably 3 years. The vessel portion of the device may be stored at between 22°-25° C. in normal atmospheric conditions for a period of preferably at least 3 years, less preferably at least 3 months, and even less preferably at least one month, and during such storage the low oxygen concentrations levels describe above will remain.

As described herein, the vessel or bag of low oxygen concentration fluid may be used to bathe, contact, or the cells to be transplanted for a sufficient time to activate the hypoxic metabolic pathway. In a preferred embodiment, the cells to be transplanted may alternatively be bathed in saline solution having normal oxygen levels and subsequently bathed in a saline solution having low oxygen concentration. This process of alternating between the two saline solutions may be repeated several times to increase the activation of the cell's hypoxic response pathway.

The invention discloses a method of using manufactured physiologic electrolyte solutions formulated to have a low concentration of oxygen and packaged to sustain the intended low concentration of oxygen to precondition cells to be transplanted to a patient. The method in a preferred embodiment comprises use of an apparatus comprising a vessel constructed such that it is substantially impermeable to oxygen gas, said vessel holding a mammalian cell contacting fluid having a hypoxic oxygen concentration between about 0.01% to about 5% of the fluid's maximal dissolved oxygen capacity and more preferably between 0.01% and 1.5% of the fluid's maximal dissolved oxygen concentration. A preferred shelf life of the hypoxic cell contacting fluid is greater than 1 year achieved by employing low oxygen permeation packaging.

For purposes of clarity, it is noted that Oxygen saturation or dissolved oxygen (DO) is a relative measure of the amount of oxygen that is dissolved or carried in a given medium. It can be measured with a dissolved oxygen probe such as an oxygen sensor. Atmospheric pressure, temperature and salinity affect DO levels. Dissolved Oxygen Percent Saturation is also important to determine because cold, fresh water holds more oxygen than warm or salty water. For example, pure water at 4° C. can hold 13.1 ppm and water at 22° C. can hold 8.7 ppm. Hereafter, the oxygen percent saturation values used are referring to oxygen saturation values at room temperature, a salinity of 0.9% NaCl solution at an elevation of sea level.

The cell-retaining chamber preferably provides a means for removal or displacement of atmospheric air that could result in rapid increase of oxygen concentration of the hypoxic cell-contacting fluid that would negatively affect the intended activation of the cell's hypoxic responsive pathway. In one example this means comprises at least one hydrophobic air vent. Examples of cell retaining chambers may include but are not limited to syringes, centrifuge canisters and filters.

The cell-contacting fluid is preferably a non-cytotoxic physiologic fluid that retains at least 90% cell viability after a 1-hour exposure. In a preferred embodiment, the hypoxic cell-contacting fluid is an electrolyte solution that is sterile and non-pyrogenic and has a chemical formulation with the notable exception of oxygen concentration that is substantially similar to a commercially available physiologic electrolyte fluid. Examples of such physiologic electrolyte solutions in commercial may include include Plasma-Lyte A manufactured by Baxter Healthcare, Deerfield, Ill., normal saline (0.90% w/v of NaCl, about 300 mOsm/L), half-normal saline (0.45% NaCl), optionally with “D5” (5% dextrose) and containing approximately 77 mEq/L of Na and Cl and 50 g/L glucose, Lactated Ringer's solution, Acetated Ringer's solution, Phosphate buffered saline (PBS), TRIS-buffered saline (TBS), Hank's balanced salt solution (HBSS), Earle's balanced salt solution (EBSS), Standard saline citrate (SSC), HEPES-buffered saline (HBS), Normosol-R and Gey's balanced salt solution (GBSS).

In the present invention, the achievement of contacting mammalian cells with a hypoxic cell-contacting fluid is not dependent on the diffusion of atmospheric gases into and out of the cell-contacting fluid, which has been shown by Newby as a slow and unpredictable process. Rather, in the present invention, the obstacles described by Newby are overcome by the invention of pre-manufactured physiologic hypoxic fluids having a low oxygen concentration and packaging the fluids in a vessel so as to stably maintain the intended hypoxic concentration desired. The disclosed method, apparatus, and composition provide a highly improved means to transiently contact living cells with a hypoxic fluid for the purpose of activating the hypoxia response pathway resulting in increased levels of cellular HIF-1α concentration with its attendant therapeutic benefits. For the purpose of the present invention, the activating the hypoxia response pathway resulting in increased levels of cellular HIF-1α concentration with its attendant therapeutic benefits is designated as cell preconditioning.

### BRIEF DESCRIPTION OF THE FIGURES

The foregoing aspects and many of the attendant advantages of the invention will become more readily appreciated as the same becomes better understood by reference to the following detailed description, when taken in conjunction with the attached charts and figures, wherein:

FIG. 1 is a simplified diagram of a preferred embodiment of the present invention;

FIG. 2 is a a simplified diagram showing an alternative embodiment of the present invention;

FIG. 3A a detailed diagram showing a first step in an alternative method of the present invention;

FIG. 3B a detailed diagram showing a second step in an alternative method of the present invention;

FIG. 3C a detailed diagram showing a third step in an alternative method of the present invention;

FIG. 4A a detailed diagram showing a fourth step in an alternative method of the present invention;

FIG. 4B a detailed diagram showing a fifth step in an alternative method of the present invention;

FIG. 5A a detailed diagram showing a sixth step in an alternative method of the present invention;

FIG. 5B a detailed diagram showing a seventh step in an alternative method of the present invention;

FIG. 6 a detailed diagram showing a eighth step in an alternative method of the present invention; and

FIG. 7 is an image of an embodiment of the present invention according to a preferred form; and

FIG. 8 is a graph showing dissolved oxygen levels in culture medium over time.

### DETAILED DESCRIPTION OF THE INVENTION

The following description is presented to enable a person of ordinary skill in the art to make and use various aspects and examples of the present invention. Descriptions of specific materials, techniques, and applications are provided only as examples. Various modifications to the examples described herein will be readily apparent to those of ordinary skill in the art, and the general principles defined herein may be applied to other examples and applications without departing from the spirit and scope of the invention. Thus, the present invention is not intended to be limited to the examples described and shown, but is to be accorded the scope consistent with the appended claims.

The preferred embodiment of the Applicant's method for cell preconditioning comprises the steps of providing a multi-compartment disposable bag set comprising a pre-filter container, a cell filter, and an effluent container in controllable liquid connection with one another. Using hoses and clamps, fluid may be directed through the system as described. It is noted that the precise nature and configuration of the disposable bag-set may vary and that any suitable materials, components, and configurations as known in the art may be substituted for the precise configuration of this preferred embodiment.

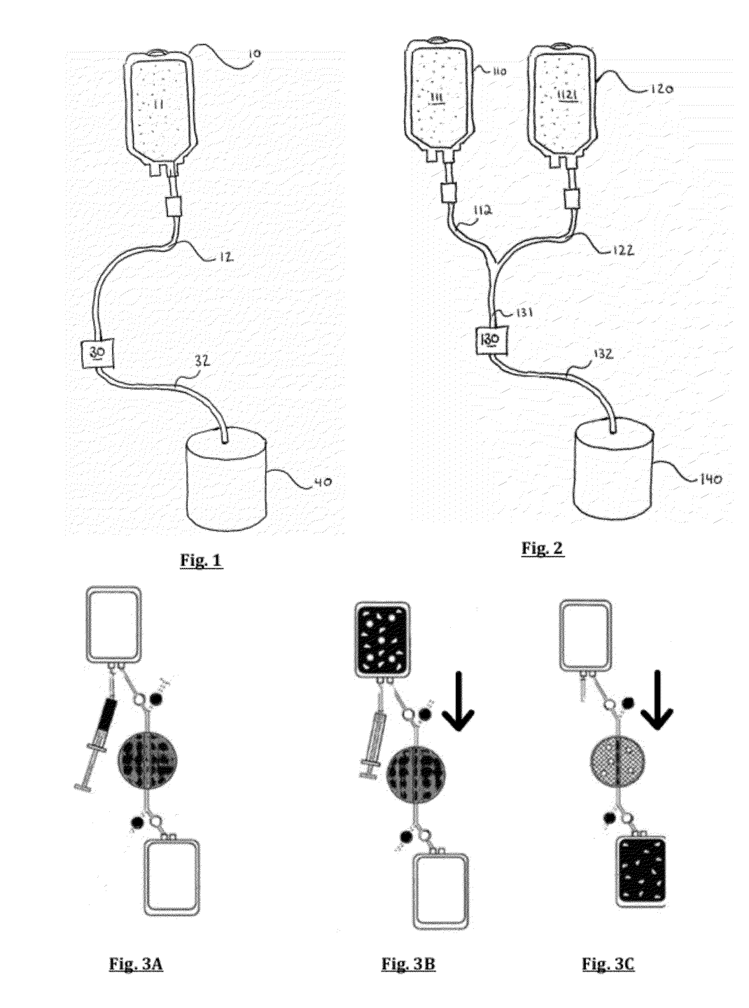

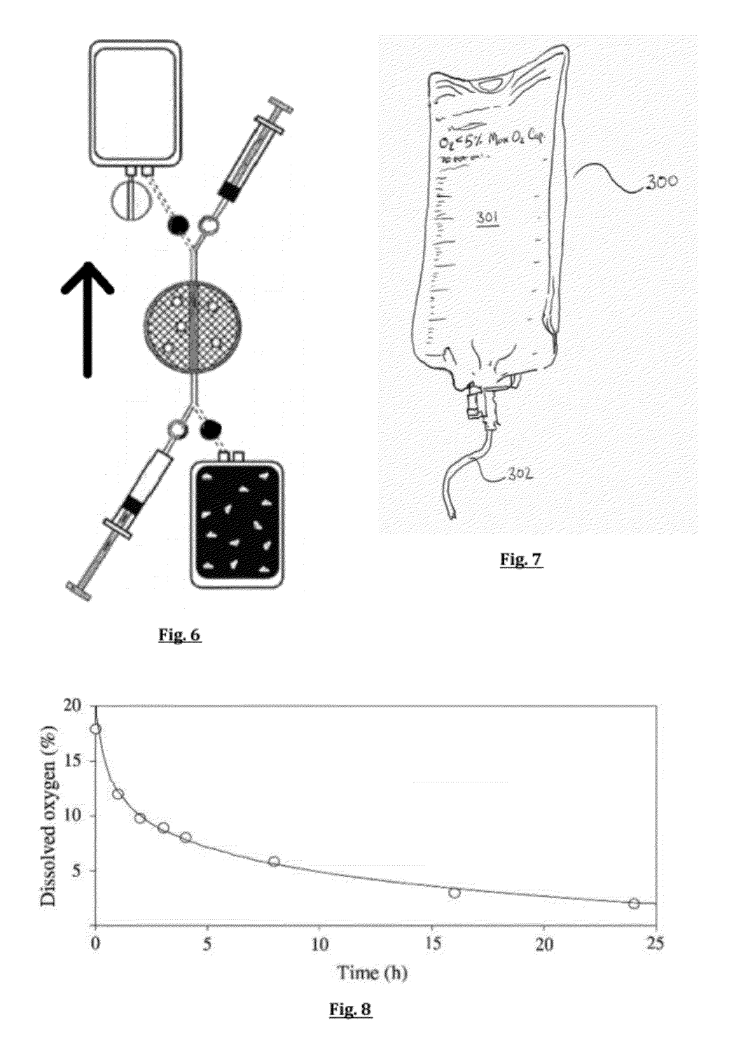

In a preferred embodiment, a diagrammatic view of an exemplary device for use with the preconditioning cell suspensions method is disclosed in its simplest form in FIG. 1. In this embodiment the device comprises a hypoxic fluid vessel 10 (also referred to herein as a “first vessel”, and also shown alone in FIG. 7) containing within it a hypoxic fluid 11. In fluid and mechanical connection thereto by a hypoxic fluid tube 12 is a mammalian cell-retaining chamber 30, and in fluid and mechanical connection thereto by a waste tube 32 is a waste chamber 40. The mammalian cell-retaining chamber comprises mammalian cell retentate. The retentate may be prepared from cell culture or be harvested from a living body. The cells prepared by this method may be derived from mammalian tissue such as but not limited to bone marrow aspirate, cord blood, peripheral blood, tissue digest, and tissue culture. The mammalian cell-retaining chamber further comprises a cell filter (not shown). Examples of suitable cell filters for this invention include but are not limited to blood leukocyte filters manufactured by Pall Corporation, Long Island, New York and Asahi Kasei Medical, Tokyo, Japan as well as cell retaining filters manufactured by Millipore, Billerica, Mass. An embodiment of the present invention uses a multicompartment filtration apparatus as taught by Sowemimo-Coker et at in U.S. Pat. No. 7,291,450.

The tubes disclosed herein allow fluid to flow from the hypoxic fluid vessel 10, down the hypoxic fluid tube 12, through the mammalian cell-retaining chamber 30, through the waste tube 32, and ultimately to the waste chamber 40. Fluid control means such as pinch clamps, stopcock valves are known in the art as means to regulate flow of such interconnected vessels and are not labeled.

Turning to FIG. 2, a diagrammatic representation of the device according to an alternative embodiment is depicted in its simplest form. Here the device again comprises a hypoxic fluid vessel 110 containing a hypoxic fluid 111, the vessel and fluid in communication through a hypoxic fluid tube 112 an via a central tube 132 with a mammalian cell retaining chamber 130. This alternative embodiment further comprises a normoxic fluid vessel 120 containing a normoxic fluid 121 in fluid connection with said mammalian cell retaining chamber 130 via a normoxic fluid tube 122 and said central tube 131 as shown.

Preferably, hypoxic fluid vessel 10/110 is substantially oxygen gas impermeable. Alternatively, hypoxic fluid vessel 10/110 is an oxygen permeable vessel but enclosed within a secondary container constructed of materials and of a quality that are substantially oxygen impermeable. Hypoxic fluid vessel 10/110 (and normoxic fluid vessel 120, if present) preferably comprise a flexible bag such as a transfer pack (e.g., Fenwal Transfusion Technologies, Lake Zurich, Ill.) or a syringe (Becton Dickinson, Franklin Lakes, N.J.). The flexible bag or syringe may be packaged in a secondary container that is designed and constructed of materials that will be airtight and promote the maintenance of the intended dissolved oxygen concentration by providing a substantially oxygen impermeable barrier. For example, sealing the flexible bag container or syringe container within a composite bag having a oxygen impermeable metal layer and a flexible elastic plastic liner bag which, when heat-sealed shut around the low oxygen containing solution, will effectively block oxygen permeation for years.

Hypoxic fluid vessel 10/110 may further comprise an oxygen-sensing tablet (such as those available by Powdertech Co. Ltd. out of Chiba, Japan), between the outer skin of the packaging and the outer wall of the vessel container to provide visual evidence that the hypoxic condition has been maintained. The vessel may further comprise an oxygen consuming chemical (e.g., iron powder) between the outer skin of the packaging and the outer wall of the vessel to provide protection against trace oxygen permeating through the wall of the outer vessel to provide for longer shelf life of the hypoxic fluid.

Hypoxic fluid vessel 10 (and normoxic fluid vessel 120, if present) preferably further comprise resistant shells, constructed to resist or avoid pinholes, cuts, or channels that tend to create pathways for oxygen containing gas exchange or leaks. It is well known that oxygen impermeable materials include glass and aluminum foil. Ethylene Vinyl Alcohol, commonly abbreviated EVOH, is a formal copolymer of ethylene and vinyl alcohol. The plastic resin primary purpose is to provide barrier properties, primarily as an oxygen barrier for improved food packaging shelf life. EVOH is typically coextruded or laminated as a thin layer between cardboard, foil, or other plastics. PVdC (Polyvinylidene Chloride) is also a highly effective barrier coating polymer. The use of multi-layer laminate of fine dimension layers, at least one of which is resistant to permeation by oxygen can be used to construct the primary container or the secondary container. An example of such a material is PAKVF4PC MylarFoil™ sold by IMPAK Corporation out of Los Angeles, Calif. In use, the Hypoxic fluid vessel 10 (and normoxic fluid vessel 120, if present) is/are designed and constructed of materials so as to substantially maintain the intended oxygen concentration of the physiologic solution. Any of these materials are suitable and may be used in the composition of the vessel.

In a preferred embodiment at least one of the cell preconditioning fluids (that is, the hypoxic fluid 11 or 111 and/or the normoxic fluid 112) has a temperature between 16° C. and 40° C. while in contact with the mammalian cells in the cell filter of the mammalian cell-retaining chamber (30 or 130). In a more preferred embodiment at least one of the cell preconditioning fluids has a temperature between 25° C. and 37° C. while in contact with the cells.

The cell preconditioning fluids may be made of or derived from commercial solutions such as Plasma-Lyte® (Electrolyte) of Baxter® Inc. of Deerfield, Ill. or Normosol-R® of Abbott Labs, North Chicago, Ill. Such solutions are currently manufactured in containers that are oxygen gas permeable, such as polyvinyl chloride plastic. Therefore, the oxygen concentration is in equilibrium with the atmosphere (˜8-9 ppm dissolved oxygen).

The hypoxic fluid preferably has a maximal dissolved oxygen capacity substantially equivalent to normal saline (approximately between 8-10 mg/L) and has an oxygen concentration between about 0% to about 5% of said maximal dissolved oxygen capacity wherein said oxygen concentration. This concentration level of oxygen remains substantially stable when the apparatus is stored at 22°-25° C. in normal atmospheric conditions for a period of at least 30 days. For purposes of this patent application normal atmospheric conditions are defined as a composition of gas comprising approximately 75-85% nitrogen and approximately 15-25% oxygen and the atmospheric pressure is substantially equivalent to atmospheric pressure at sea level Normal saline is an aqueous solution of 0.9% sodium chloride (weight/volume). The cell-contacting fluid is preferably a non-cytotoxic physiologic fluid that retains at least 90% cell viability after a 1-hour exposure. In a preferred embodiment, the hypoxic cell-contacting fluid is an electrolyte solution that is sterile and non-pyrogenic and has a chemical formulation with the notable exception of oxygen concentration that is substantially similar to a commercially available physiologic electrolyte fluid. Examples of such physiologic electrolyte solutions in commercial may include Plasma-Lyte A manufactured by Baxter Healthcare, Deerfield, Ill., normal saline (0.90% w/v of NaCl, about 300 mOsm/L), half-normal saline (0.45% NaCl), optionally with “D5” (5% dextrose) and containing approximately 77 mEq/L of Na and Cl and 50 g/L glucose, Lactated Ringer's solution, Acetated Ringer's solution, Phosphate buffered saline (PBS), TRIS-buffered saline (TBS), Hank's balanced salt solution (HBSS), Earle's balanced salt solution (EBSS), Standard saline citrate (SSC), HEPES-buffered saline (HBS), Normosol-R and Gey's balanced salt solution (GBSS).

Continuing with FIG. 2, and as with the previous embodiment shown in FIG. 1, said mammalian cell retaining chamber 130 is in fluid connection with waste chamber 140 via a waste tube 132. As with the preferred embodiment, all components are connected so as to create a continuous fluid path via the tubing as shown. Fluid control means such as pinch clamps, stopcock valves are known in the art as means to regulate flow of such interconnected vessels and are not labeled. It is notable that for purposes of this patent application any amount above 10% dissolved oxygen of the maximum dissolved oxygen concentration for a fluid is considered be a high oxygen concentration and normoxic.

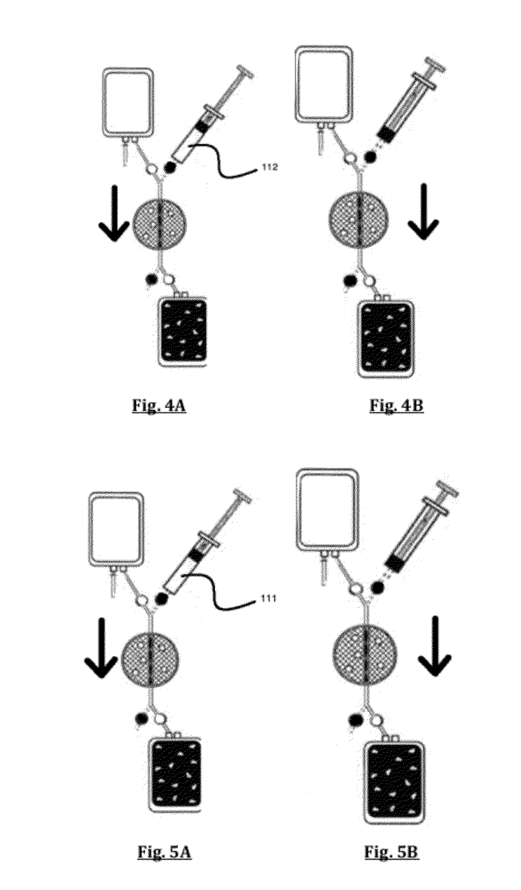

A more detailed drawing of the multi-compartment disposable bag system first outlined in FIG. 2 is shown in FIG. 3. Because FIGS. 3A-6 are intended to show the method of movement of fluid through the system in accordance with an alternative embodiment of the invention, items listed are but for a two exceptions not labeled. Here, the system comprises a syringe attached to an input syringe port which is located upstream of and in fluid communication with a pre-filter, which in turn is located upstream of and in fluid communication with a cell filter, which in turn is located upstream of and in fluid communication with an effluent container. The system comprises clamps between the pre-filter container, the cell filter, and the filter effluent container. These clamps are initially open. The system further comprises at least two additional syringe ports, one located upstream and in fluid communication with the cell filter, one located downstream and in fluid communication with the cell filter. Both additional syringe ports are shown with a solid black circle, indicating at this point in the process the syringe ports are closed. It is noted that the use of a syringe and syringe ports is not required, other devices known in the art such as pumps may be readily substituted, in this and in all steps of the method. The syringe shown in at FIG. 3A contains mammalian cells.

Continuing to FIG. 3B, the depiction shows the suspended mammalian cells having been ejected from the syringe and into the pre-filter container. Here the suspended mammalian cells may be directed to the cell filter, after they pass through the pre-filter container. Preferably at this step the valve, clamp or other means of impeding a fluid connection between the components is open, as shown by a solid white circle between the pre-filter container and the filter, and again between the filer and the now-empty waste chamber. Moving along to FIG. 3C, the arrow present in the image indicates the general movement direction of liquid flow. After the liquid flows through the system to the waste chamber, the mammalian cells remain suspended as a filter retentate in the cell filter, the remaining solution having passed through the cell filter and into the waste chamber. At this point the syringe may be removed from the system. In one embodiment the pre-filter is adapted to remove at least one of leukocytes or stem cells from the cell suspension.

Turning now to FIG. 4A, a diagrammatic representation of the next step in a preferred embodiment of the method is shown. As is diagrammed, a syringe of a normoxic fluid 112 has been attached to a syringe port upstream of the cell filter (on the same side of the cell filter as the pre-filter), and the clamps between that syringe port, the cell filter, and the filter effluent container have been opened. In a preferred embodiment the first normoxic fluid 112 is an oxygen rich contacting fluid, i.e. a normoxic contacting fluid. Next at FIG. 4B, after attachment, the normoxic fluid 112 is ejected from the syringe and contacts substantially all the suspended cells contained within the cell filter. The clamps between the syringe, the cell filter, and the filter effluent container may be may be manipulated so that the cells in the cell filter remain in contact with the normoxic fluid 112 for a first incubation time which is preferably between 5 minutes and 2 hours. Excess fluid passes through to the waste chamber.

Turning next to FIG. 5A, a syringe of a hypoxic fluid 111 is attached to a syringe port upstream of the cell filter (on the same side of the cell filter as the pre-filter), and the clamps between that syringe port, the cell filter, and the filter effluent container have been opened. In a preferred embodiment the hypoxic fluid 111 is an oxygen poor fluid, i.e. a hypoxic contacting fluid. FIG. 5B depicts the state of the system after attachment, where the hypoxic fluid 111 is ejected from the syringe and contacts substantially all the suspended cells contained within the cell filter, this step may be thought of as exchanging hypoxic fluid 111 for normoxic fluid 112. The clamps between the syringe, the cell filter, and the filter effluent container may be may be manipulated so that the cells in the cell filter remain in contact with the hypoxic fluid 111 for a second incubation time. In a preferred embodiment the second incubation time is between 1 minute and 24 hours, and is sufficient to activate cell hypoxia.

In a preferred embodiment, when the hypoxic fluid 111 is ejected from the syringe and into the cell filter the oxygen concentration of the fluid contacting the cells in the cell filter is reduced by more than 50% within 5 minutes. In a second preferred embodiment when the hypoxic fluid 111 is ejected from the syringe and into the cell filter the oxygen concentration of the fluid contacting the cells in the cell filter is reduced by more than 50% within 1 minute. In an alternative embodiment when the hypoxic fluid 111 is injected into the cell filter the oxygen concentration of the fluid contacting the cells in the cell filter is reduced by more than 50% within 5 minutes.

In a preferred embodiment the normoxic fluid 112 has a dissolved oxygen content greater than 4 mg/L, and the hypoxic fluid 111 has a dissolved oxygen content less than 3 mg/L. In a more preferred embodiment the hypoxic fluid 111 has a dissolved oxygen content of less than 2 mg/L.

In an alternative embodiment the above described steps of contacting substantially all the suspended cells with normoxic fluid 112 followed by contacting substantially all the suspended cells with hypoxic fluid 111 may be repeated two or more times. During such repetition, in one embodiment the concentration of dissolved oxygen contacting the cells retained in the cell filter is increased by at least 50% within 5 minutes, and then decreased by at least 50% within 5 minutes.

FIG. 6 represents the final step of the preferred embodiment of the method. In this step a syringe of recovery solution is attached to the previously unused syringe port located downstream of the cell filter (on the same side of the cell filter as the waste chamber), and an empty syringe is attached to the upstream syringe port. The clamps between the pre-filter, the cell filter, and the filter effluent container are closed and the clamps between the cell filter and the two syringes are opened. The recovery solution is ejected from the downstream syringe, and this recovery solution back-flushes the cell filter into the empty syringe attached to the upstream syringe port, as shown by the upwardly pointing arrow. At the completion of this step the initially empty syringe attached to the upstream syringe port preferably contains substantially all of the now preconditioned cells, which were initially (at the start of this step) located in the cell filter.

In a preferred embodiment after being preconditioned for further use, the preconditioned cells recovered from the cell filter exhibit increased levels of HIF-1α1 within 6 hours after recovery relative to cells that were not treated in accordance with the present invention. In a preferred embodiment a portion of the preconditioned cells thus recovered have increased expression of CXCR4 protein on a cell surface within 6 hours after recovery as compared to cells that were not treated in accordance with the present invention.

As described herein, the vessel or bag of low oxygen concentration fluid may be used to bathe, contact, or the cells to be transplanted for a sufficient time to activate the hypoxic metabolic pathway. In a preferred embodiment, the cells to be transplanted may alternatively be bathed in saline solution having normal oxygen levels and subsequently bathed in a saline solution having low oxygen concentration. This process of alternating between the two saline solutions may be repeated several times to increase the activation of the cell's hypoxic response pathway.

As a second exemplary method of the present invention, a solution for problems inherent in the transplantation of adipose tissue fragments (fat grafting) is presented. In this medical procedure, a surgical procedure known as lipoplasty is performed to extract adipose tissue fragments from the body. Conventionally, the adipose tissue fragments are concentrated and re-injected to the site of a cosmetic defect. The unmet need is that the transplanted adipocytes in the tissue fragment have limited survival and the intended improvement in cosmetic appearance is not sustained as desired due to cell death of the transplanted cells. There is thus a need to improve the transplanted cell survival rate so at to sustain the intended cosmetic benefit. The present invention enables this goal to be achieved in a simple and practical manner. By way of this exemplary method, the adipose tissue fragments are collected into a syringe and any air in the syringe is removed. The syringe is placed in an upright position with the needle aperture point down. After allowing the adipose tissue to float to the top, excess aqueous fluid is removed by depressing the plunger. Next, hypoxic saline solution is drawn into the syringe without introducing ambient air. The adipose tissue fragments are bathed in the hypoxic solution. The hypoxic saline solution may be replaced one or more times. The required duration of bathing the adipose tissue fragments may be determined experimentally by measuring the cellular level of HIF-1α. The intended increase in cellular level of HIF-1α is greater than two fold as compared to HIF-1α levels in untreated control cells. In a preferred embodiment, the adipose tissue fragments are bathed alternatively with low oxygen concentration saline and normal oxygen concentration saline. After preconditioning is completed, the adipose tissue fragments are returned to a living body yielding an improved therapeutic or cosmetic effect as compared to that found in the prior art.

By way of a third exemplary case, a cell suspension may be captured on a cell-retaining filter such as a leukofilter such as those used in the blood banking industry. The cells retained on the filter may be bathed with a low oxygen concentration saline solution so as to activate the hypoxic response pathway. The cells retained on the filter in a preferred embodiment of the invention are alternatively exposed to saline solutions having low oxygen concentration and normal oxygen concentration. The cells may be recovered by back-flushing the filter as described in U.S. Pat. No. 7,291,450. The required duration of bathing the adipose tissue fragments is determined experimentally by measuring the cellular level of HIF-1α. In order to precondition stem cells, the present invention teaches to transiently expose adult stem cells to cycles of both low and normal oxygen levels in a repetitive manner. In a preferred embodiment, sterile physiologic saline solutions are formulated with either a low oxygen concentration or normal oxygen concentrations. The cells are loaded on to a biomedical filter to hold them in place while they are being contacted by the saline solutions being pumped alternatively through the filter. The amount of fluid and duration of exposure to each solution is experimentally determined by measuring the amount of increased cellular HIF-1α that is generated for a particular cell type. The intended increase in cellular level of HIF-1 α is greater than two fold. One cycle is considered to be these four steps: pumping low oxygen cell contacting fluid to displace the high oxygen concentration fluid, incubation period of cells with low oxygen cell contacting fluid, pumping high oxygen cell contacting fluid over cells to displace low oxygen concentration fluid, and incubation period of cells with the high oxygen cell contacting fluid. The process is repeated for a number cycles depending upon the cell population to be treated so as to achieve the intended activation of the hypoxic response pathway as detected by measuring increasing HIF-1 α concentration in the preconditioned cells. After preconditioning is completed, the cells are injected to a living body for the purpose of achieving a therapeutic or cosmetic effect.

By way of a fourth example, a preconditioned stem cell concentrate is prepared using the steps comprising: providing a combined physiological cell contacting fluid comprising a stem cell population derived from peripheral blood, bone marrow, cord blood, expanded cell population by cell culture or adipose tissue cells or combinations thereof, and an initial cell contacting fluid that is normoxic. The mixture is passed through a cell retaining filter housing having at least one inlet and one outlet; producing a cell retentate on the filter media wherein the waste fluid exits the cell retaining chamber, subjecting the cells retained in the filter to sufficient hypoxic cell contacting fluid such that the normoxic cell contacting fluid is discharged out of the housing and substantially replaced with the hypoxic cell contacting fluid, incubating the cells with the hypoxic cell contacting fluid for a sufficient period to activate the hypoxic metabolic pathway as evidenced by subsequent increased cellular HIF-la protein levels, and finally removing the cell retentate from the housing to produce a preconditioned cell concentrate. The cell retaining housing may be a filter such as those sold by Pall Corporation, Long Island, N.Y. or Asahi Corp., Tokyo, Japan. The process of alternating the contacting fluid between hypoxic and normoxic cell contacting fluids may be carried out as many times as required to achieve the intended biological effect of activating the hypoxic metabolic pathway. The intended increase in cellular level of HIF-1 α is greater than two fold. It is preferable to have at least two exchanges with at least a 5-minute incubation between fluid exchanges.

The present invention enables donor cell preconditioning to be performed at the point of care. The preconditioning of the cells at the point of care within the same surgical procedure is a significant advancement in regenerative medicine. From a patient perspective, injection of stem cells into ischemic tissue is extremely painful. If the entire process of harvesting, conditioning and delivering the cells within the same surgical procedure can be accomplished, the cells may be injected under anesthesia with no discomfort to the patient. From an economic perspective, the cost of preconditioning the cells will be dramatically reduced as the movement of the cells into a GMP laboratory for processing is inherently expensive due to the cost associated with maintaining a GMP environment and the expert staff employed therein. Reducing human error in the donor cell conditioning process is another advantage of this invention. It simplifies the most critical parts of the process to ensure reproducibility and reliability of performance by providing pre-manufactured oxygen concentration controlled fluids that are ready on demand for clinical use.

Although the invention has been shown and described with respect to certain embodiments, it is obvious that equivalent alterations and modifications will occur to others skilled in the art upon the reading and understanding of the specification. In particular, with regard to the various functions performed by the above-described components, the terms (including any reference to a “means”) used to describe such components are intended to correspond, unless otherwise indicated, to any component which performs the specified function of the described component (e.g., that is functionally equivalent) even though not structurally equivalent to the disclosed component which performs the functions in the herein exemplary embodiments of the invention. In addition, while a particular feature of the invention may have been disclosed with respect to only one embodiment, such feature may be combined with one or more other features of other embodiments as may be desired or advantageous for any given or particular application.

1. A method for the preparation of mammalian cells for implantation into a living body, the method comprising the steps of:
a. providing an apparatus comprising:
i. a first vessel substantially impermeable to gaseous oxygen;
ii. hypoxic fluid within said first vessel, the hypoxic fluid having a maximal dissolved oxygen capacity and having an oxygen concentration level of not more than 5% of said maximal dissolved oxygen capacity; and
iii. a mammalian cell retaining chamber comprising a filter and mammalian cells having a level of HIF-1α; and
b. contacting said hypoxic fluid with said mammalian cells within said filter.

2. The method of claim 1 wherein said oxygen concentration level is between 0.05% and 1.5% of said maximal dissolved oxygen capacity.

3. The method of claim 1 wherein said hypoxic fluid within said first vessel maintains said oxygen concentration level when said first vessel is stored at 22°-25° C. in normal atmospheric conditions for a period of at least 30 days.

4. The method of claim 1 further comprising storing said first vessel at 22°-25° C. in normal atmospheric conditions for a period of at least 30 days such that said oxygen concentration of not more than 5% of said maximal dissolved oxygen capacity is maintained in said hypoxic fluid.

5. The method of claim 1 wherein said hypoxic fluid is a physiologic electrolyte solution.

6. The method of claim 1 wherein said contacting step is of sufficient duration to result in an in an increase in said level of HIF-1α.

7. The method of claim 1 further comprising implanting said mammalian cells into a living body subsequent to said contacting step.

8. The method of claim 1 wherein said mammalian cells are selected from the group consisting of: whole blood, whole blood fractions, buffy coat fractions, platelet rich plasma fractions, granulocyte concentrates, bone marrow aspirate, cord blood, lipoaspirate, mammalian tissue fragments, adipose tissue fragments, umbilical cord tissue fragments, mesenchymal stem cells, hematopoietic stem cells, progenitor cells, induced pluripotent stem cells, tissue cultured expanded cells, mammalian tissue fragments bound by a fibrin containing scaffold, mammalian cell suspension bound by a fibrin containing mesenchymal stem matrix, nucleated cells bound to an extra-cellular matrix, and mesenchymal stem cells bound to a bone graft matrix.

9. The method of claim 8 further comprising implanting said mammalian cells into a living body subsequent to said contacting step.

10. The method of claim 1 wherein said apparatus further comprises:
a. a second vessel containing a normoxic fluid; and
b. wherein said mammalian cell retaining chamber is fluidly connected to said first vessel and said second vessel.

11. The method of claim 10 further comprising contacting said mammalian cells alternatingly with said normoxic fluid and said hypoxic fluid.

12. The method of claim 11 wherein said contacting steps are of sufficient duration to result in an in an increase in said level of HIF-1α.

13. A vessel for storing fluid for use in increasing the level of HIF-1α in mammalian cells, the vessel comprising:
a. an outer skin substantially impermeable to gaseous oxygen;
b. an inner volume within said outer skin; and
c. hypoxic fluid in said inner volume, the hypoxic fluid having a maximal dissolved oxygen capacity and an oxygen concentration level of not more than 5% of said maximal dissolved oxygen capacity.

14. The vessel according to claim 13 wherein said hypoxic fluid substantially fills said inner volume.

15. The vessel according to claim 13 wherein substantially no oxygen gas is within said inner volume.

16. The vessel according to claim 13 wherein said maximal dissolved oxygen capacity is between 8 and 10 mg/L.

17. The vessel according to claim 13 further comprising an oxygen-sensing tablet between the outer skin of the packaging and the outer wall of the vessel container to provide visual evidence that the hypoxic condition has been maintained.

18. The vessel according to claim 13 further comprising an oxygen consuming chemical between the outer skin of the packaging and the outer wall of the vessel to provide protection against trace oxygen permeating through the wall of the outer vessel to provide for longer shelf life of the hypoxic fluid.

19. A method for the preparation of mammalian cells for implantation into a living body, the method comprising the steps of:
a. providing an apparatus comprising:
i. a first vessel substantially impermeable to gaseous oxygen;
ii. hypoxic fluid within said first vessel, the hypoxic fluid having a maximal dissolved oxygen capacity and having an oxygen concentration level of not more than 5% of said maximal dissolved oxygen capacity; and
iii. a mammalian cell retaining chamber comprising mammalian cells having a level of HIF-1α; and
b. contacting said hypoxic fluid with said mammalian cells.

20. The method of claim 1 wherein said hypoxic fluid within said first vessel maintains said oxygen concentration level when said first vessel is stored at 22°-25° C. in normal atmospheric conditions for a period of at least 30 days.
